(19) United States
(12) Patent Application Publication
(10) Pub. No.: US 2005/0288566 A1
(43) Pub. Date: Dec. 29, 2005

(54) Apparatus with partially insulated needle for measuring tissue impedance

(76) Inventors: Joseph A. Levendusky, Groton, MA (US); Robert F. Rioux, Ashland, MA (US); Kurt A.E. Geitz, Sudbury, MA (US); Scott A. Dodson, Franklin, MA (US); Robert M. Rauker, Chester Springs, PA (US)

Correspondence Address: Edward N. Bachand;DORSEY & WHITNEY LLP, Suite 3400, 4 Embarcadero Center, San Francisco, CA, 94111, US

(21) Appl. No.: 11/120,436

(22) Filed: May 2, 2005

Related U.S. Application Data
(60) Provisional application No. 60/567,199, filed on Apr. 30, 2004.

(51) Int. Cl. A61B 5/04

(52) U.S. Cl. 600/372; 607/101

(57) ABSTRACT

An apparatus for use with a radio frequency generator and an indifferent electrode to treat tissue in a mammalian body includes a needle having a distal portion and being adapted for coupling to the radio frequency generator and a layer of insulating material extending around the needle but exposing a part of the distal portion of the needle. The exposed part of the needle is sized as a function of the thicknesses of the plurality of layers of tissue to facilitate placement of the distal portion of the needle in the plurality of layers by measuring impedance between the needle and the indifferent electrode.

### CROSS REFERENCE TO RELATED APPLICATION

This application claims priority to U.S. provisional patent application Ser. No. 60/567,199 filed Apr. 30, 2004, the entire content of which is incorporated herein by this reference.

### SCOPE OF THE INVENTION

The present invention relates to medical devices and methods for treating a mammalian body and more particularly to medical devices and methods having injection needles.

### BACKGROUND

Medical devices have been provided for the delivery of a material to various portions of a wall forming a vessel such as the gastrointestinal tract of a mammalian body to create implants in the wall. See, for example, U.S. Pat. No. 6,251,063. Apparatus, such as disclosed in U.S. Pat. No. 6,358,197, have been provided for enhancing the consistent formation of such implants.

In connection with routine hypodermic injections, apparatus have been provided to sense a hypodermic needle's transition between various subcutaneous tissue types. See, for example, U.S. Pat. No. 5,271,413. Such apparatus, however, have not been disclosed for use in endoluminal applications where the thickness of tissue layers is very small.

### SUMMARY OF THE INVENTION

An apparatus for use with a radio frequency generator and an indifferent electrode to treat tissue having a plurality of layers with respective thicknesses in a mammalian body is provided. The apparatus includes a needle having a distal portion and being adapted for coupling to the radio frequency generator and a layer of insulating material extending around the needle but exposing a part of the distal portion of the needle. The exposed part of the needle is sized as a function of the thicknesses of the plurality of layers of tissue to facilitate placement of the distal portion of the needle in the plurality of layers by measuring impedance between the needle and the indifferent electrode. A method for treating tissue having a plurality of layers is provided.

### BRIEF DESCRIPTION OF THE DRAWINGS

The accompanying drawings, which are somewhat schematic in some instances and are incorporated in and form a part of this specification, illustrate several embodiments of the invention and, together with the description, serve to explain the principles of the invention.

FIG. 1 is an elevational view of an apparatus of the present invention.

FIG. 2 is an elevational view of a portion of the distal extremity of the apparatus of FIG. 1 taken along the line 2-2 of FIG. 1.

FIG. 3 is a plan view of a kit, somewhat schematic and partially cut away, including portions of the apparatus of FIG. 1 for treating the upper portion of the gastrointestinal tract in accordance with the method of the present invention.

FIG. 4 is an elevational view of a portion of the apparatus of FIG. 1 in a portion of a passageway of a mammalian body.

FIG. 5 is an enlarged elevational view of a portion of the apparatus of FIG. 1 in a portion of the passageway of the mammalian body of FIG. 4.

FIG. 6 is a further enlarged view of the distal portion of the apparatus of FIG. 1 penetrating tissue in a passageway of the mammalian body of FIG. 4.

### DESCRIPTION OF THE INVENTION

Apparatus of the type disclosed in U.S. Pat. No. 6,251,063, the entire content of which is incorporated herein by this reference, have been utilized for treating the wall of an internal cavity of a mammalian body accessible by a natural body opening. An exemplary cavity is the gastrointestinal tract of a human accessible from the mouth and formed by a wall having a plurality of layers of tissue. An exemplary apparatus or medical device 21 is shown in FIG. 1 and includes a probe member or probe 22 having an optical viewing device 23. A needle assembly or injection device 26 is slidably disposed or carried by probe 22. Treatment device 21 further includes a supply assembly 27 and a radio frequency supply and controller 28 coupled to the proximal end portion of needle assembly 26.

A conventional or other suitable gastroscope or endoscope can be used for probe 22. The exemplary probe 22 includes a flexible elongate tubular member or insertion tube 31 having proximal and distal extremities 31a and 31b and a distal face 32. Insertion tube 31 has been sectioned in FIG. 1 so that only a portion of proximal extremity 31a and distal extremity 31b are shown. A handle means or assembly is coupled to proximal extremity 31a of the insertion tube 31 and includes a conventional handle 33. The tubular insertion tube 31 is provided with at least one bore and preferably a plurality of bores or passageways 36 extending from proximal extremity 31a to distal extremity 31b. A portion of one such passageway 36 is shown in FIG. 1.

Optical viewing device 23 is formed integral with probe 22 and has an optical element or objective lens (not shown) carried by one of the passageways 36 of the device 23. The objective lens has a field of view at distal face 32, which permits the operator to view forwardly of insertion tube distal extremity 31b. Optical viewing device 23 further includes an eyepiece 41 mounted on the proximal end of handle 33. A connection cable 42, a portion of which is shown in FIG. 1, extends from handle 33 to a conventional light source 43. At least one light guide extends through cable 42 and insertion tube 31 for providing illumination forwardly of distal face 32 of the insertion tube 31.

One of the passageways provided in insertion tube 31 extends to a side port 46 formed in handle 33. Insertion tube 31 is flexible so as to facilitate its insertion and advancement through a body and is provided with a bendable distal end for selectively directing distal face 32 in a desired direction. A plurality of finger operable controls 47 are provided on handle 33 for, among other things, operating the bendable distal end of insertion tube 31 and the supply and removal of fluids through the insertion tube 31.

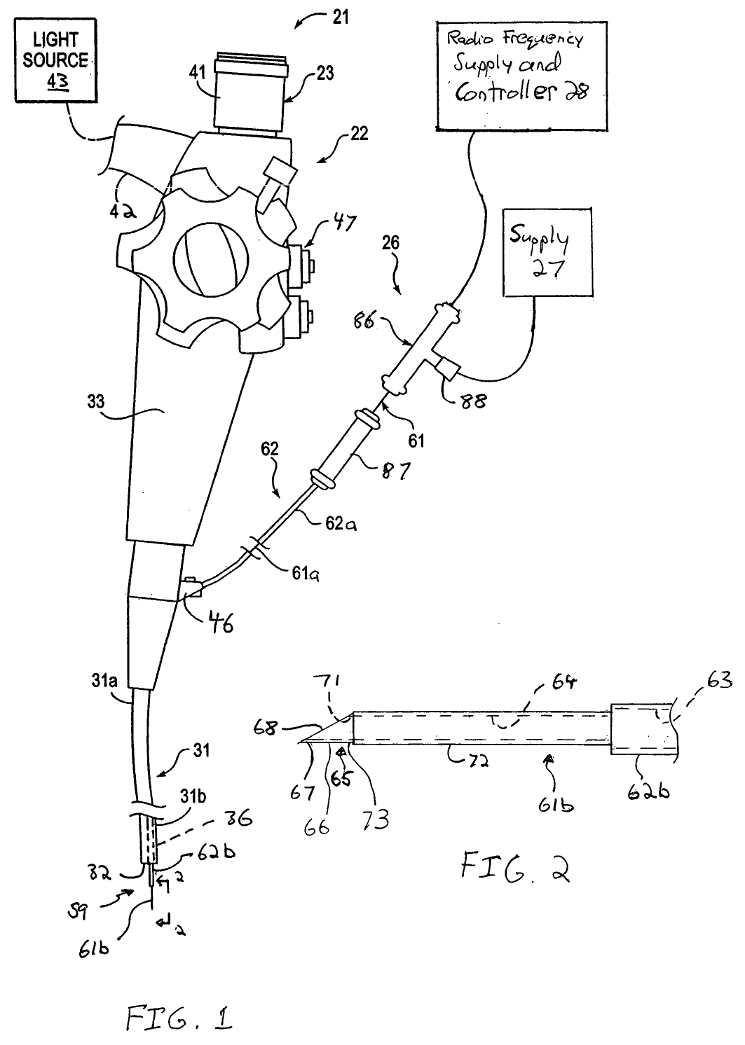

Injection device 26 includes a stylet 59 having a needle member 61 provided with a proximal end portion or extremity 61a and a distal end portion or extremity 61b and an optional sleeve member or sleeve 62 provided with a proximal end portion or extremity 62a and a distal end portion or extremity 62b. (See FIGS. 1-2) Sleeve or first elongate tubular member 62 is made from any suitable material such as flexible plastic or metal and has a lumen 63 extending longitudinally therethrough for receiving the needle or second tubular member 61. The sleeve 62 and the needle member 61 are slidable relative to each other in a longitudinal direction. In this regard, needle member 61 is slidably disposed in lumen 63 of sleeve 62, partially illustrated in FIG. 2, and movable between a retracted position in which the needle member is recessed within distal end portion 62b of sleeve and an extended position in which the needle member 61 projects distally of the sleeve 62. Needle member 61 and sleeve 62 can be slidably disposed within a passageway 36 and side port 62 of insertion tube 31 and each have a length so that when distal end portions 61b and 62b are extending from distal extremity 31b of the insertion tube 31 or otherwise in the vicinity of distal face 32, proximal end portions 61a and 62a are accessible at side port 46.

The hollow or tubular needle member 61 has a lumen or passage 64 extending longitudinally therethrough from proximal end portion 61a to distal end portion 61b (see FIG. 2). In one embodiment of injection device 26, the proximal portion 61a of the needle member is made from flexible plastic tubing and the distal extremity 61b of the needle member is a slender tube or needle 65 made from a suitable conductive material such as metal and preferably stainless steel or a shape memory allow. In another embodiment, the entire needle member 61, including needle 65 thereof, is made from a suitable conductive material such as metal and preferably stainless steel or a shape memory allow. The needle 65 is preferably circular in cross section and has a transverse dimension or diameter ranging from 0.5 to 3.0 millimeters and preferably approximately 0.5 millimeters. As shown most clearly in FIG. 2, the needle 65 is formed by a cylindrical wall 66 and has a sharpened or beveled distal tip 67 formed in part by an end surface 68 preferably lying in a plane extending at an oblique angle to the longitudinal axis of the needle 65. At least one opening 71 is provided in needle 65 and can provided in the cylindrical wall 66 or end surface 63 of the needle 65 for communicating with passageway 66. In one preferred embodiment, illustrated in the drawings, the opening 71 is provided in the end surface 68. It is appreciated that a plurality of openings 71 can be provided in needle 65, for example in the wall 66 and end surface 68.

At least a portion of needle 65 is coated or otherwise covered with a layer 72 of a suitable insulating material such as plastic and preferably a heat shrink thermoplastic elastomer, a dip-coated or spray-coated thermoset or an interference-fit tubing. More specific materials for layer 72 include polytetrafluoroethylene (PTFE), which can be applied for example as a spray coat, a heat shrink coating or an interference-fit tubing, and polyethylene, which can be applied for example as a heat shrink coating or an interference-fit tubing. The layer 72 is of any suitable thickness, for example ranging from 0.013 to 0.051 millimeters. A distal portion 73 of the needle is not covered by layer 72 and thus exposed (see FIG. 2). Such exposed portion or part 73 preferably includes opening 71 so that when needle 65 is introduced into tissue for injecting material therein, the exposed portion 73 of the needle 65 is located in the tissue near the material being injected in the tissue. In one embodiment (not shown), layer 72 is included on the distal end of the needle 65 so that exposed portion 73 is provided proximal of such portion of layer 72 and proximal of distal tip 67. In such embodiment, opening 71 is provided in exposed portion 73 and thus located proximal of a portion of layer 72 and distal tip 67. In the preferred embodiment illustrated in FIG. 2, exposed portion 73 is provided at the distal end of needle 65

The size and configuration of the exposed part 73 of needle 65 can vary. The length of the exposed part or portion 73 can be tailored to the thickness of the layers and structure of the tissue into which needle 65 is to be introduced. For example, the length of the exposed portion 73 in many applications is a function of tissue thickness. In this regard, the length or dimension of the exposed portion 73 of the needle 65 is preferably shorter or less than the thinnest layer of tissue into which material is to be injected. In a preferred embodiment, the exposed portion 73 of needle 65 has a length ranging from 0.2 to 2.0 millimeters, preferably ranging from 0.4 to 0.6 millimeters and more preferably approximately 0.5 millimeters.

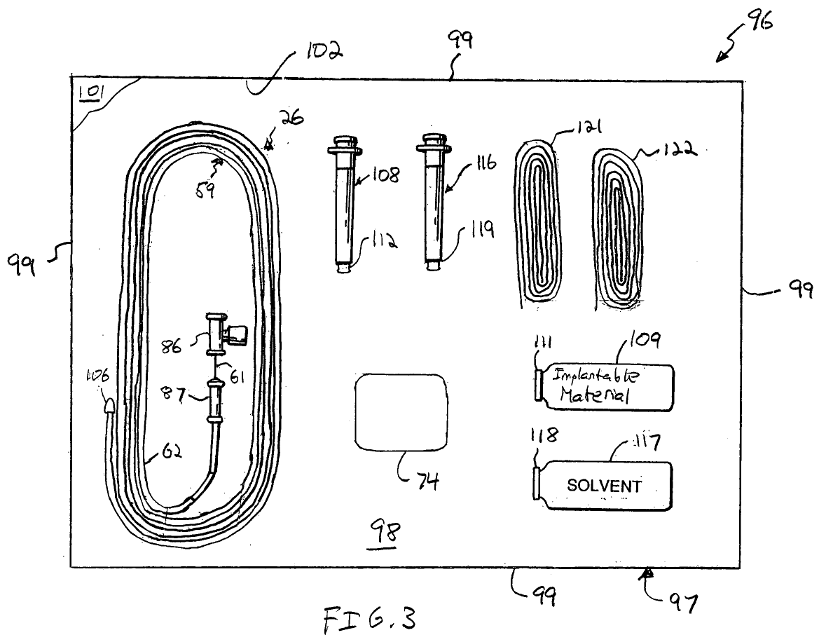

Radio frequency supply and controller 28 can be of any suitable type, and for example can include the RF 3000 Generator made by Boston Scientific Corporation of Natick, Mass. that has a 2000 W capacity and an impedance-based feedback system. Controller 28 is coupled to the proximal end portion of needle member 61 and is further coupled to a suitable indifferent or grounding electrode such as grounding pad 74 (See FIGS. 1 and 3).

In one embodiment of the injection device 26, a fluid connector 86 is secured or coupled to proximal end portion 61a of needle member 61 and a gripping member or grip 87 is secured to the proximal end portion 62a of the sleeve 62 (see FIG. 1). Fluid connector 86 includes at least one luer-fitting portion 88, or any other suitable fitting portion, which communicates with the passageway 64 in needle 61. Supply or reservoir 27 is coupled to the proximal extremity of injection device 26, and preferably to the proximal extremity 61 a of needle member 61, and can be of any suitable type. For example, one or more syringes (not shown) for containing an implantable or injectable material, or the ingredients thereof, of the present invention can be included in supply 27. The supply 27 is included within the means of medical or treatment device 21 for introducing at least one liquid, solution, composition or material through passage 64 of needle 61 and out one or more of the openings 71 provided in the distal extremity 61b of needle member 61.

Fluid connector 86 and grip 87 are longitudinally movable relative to each other so as to cause relative longitudinal movement between needle member 61 and sleeve 62. More specifically, grip 87 can be slid forwardly and rearwardly on proximal end portion 61a of the needle 61 relative to fluid connector 86. Movement of grip 87 forwardly relative to fluid connector 86 causes distal end portion 62b of sleeve 62 to extend fully over distal end portion 61b of the needle member 61 so that the needle has fully retracted within sleeve 62. Conversely, movement of grip 87 rearwardly relative to fluid connector 86 causes sleeve distal end portion 62b to retract relative to needle distal end portion 61b so as to expose needle 65 of distal end portion 61b.

Exemplary implantable materials or compositions which can be included in supply 27 and thus utilized in the method and apparatus of the present invention include any suitable material or composition from which an implant can be formed or provided, for example when a fluid, separately or in conjunction with another fluid, is introduced into the tissue of a body. The implantable material hereof includes implant-forming materials, injectable materials and solutions. Although aqueous or nonaqueous solutions are among the fluids that can be used, an inert, nonresorbable material is preferred. Preferred nonaqueous solutions are any of the solutions disclosed in International Application No. PCT/US99/29427 filed Dec. 10, 1999, the entire content of which is incorporated herein by this reference. One such implantable material comprises at least one solution which when introduced into the body forms a nonbiodegradable solid. As used herein, a solid means any substance that does not flow perceptibly under moderate stress, has a definite capacity for resisting forces which tend to deform it (such as compression, tension and strain) and under ordinary conditions retains a definite size and shape; such a solid includes, without limitation, spongy and/or porous substances. One such embodiment of the at least one solution is first and second solutions which when combined in the body form the nonbiodegradable solid. Another such embodiment is a solution which can be introduced into the body as a liquid and from which a solid thereafter precipitates or otherwise forms. A preferred embodiment of such a solution is a solution of a biocompatible composition and an optional biocompatible solvent which can further optionally include a contrast agent for facilitating visualization of the solution in the body. The solution can be aqueous or nonaqueous. Exemplary biocompatible compositions include biocompatible prepolymers and biocompatible polymers.

A particularly preferred implant forming solution is a composition comprising from about 2.5 to about 8.0 weight percent of a biocompatible polymer, from about 52 to about 87.5 weight percent of a biocompatible solvent and optionally from about 10 to about 40 weight percent of a biocompatible contrast agent having a preferred average particle size of about 10 μm or less. It should be appreciated that any percents stated herein which include a contrast agent would be proportionally adjusted when the contrast agent is not utilized. Any contrast agent is preferably a water insoluble biocompatible contrast agent. The weight percent of the polymer, contrast agent and biocompatible solvent is based on the total weight of the complete composition. In a preferred embodiment, the water insoluble, biocompatible contrast agent is selected from the group consisting of barium sulfate, tantalum powder and tantalum oxide. In still a further preferred embodiment, the biocompatible solvent is dimethylsulfoxide (DMSO), ethanol, ethyl lactate or acetone.

The term “biocompatible polymer” refers to polymers which, in the amounts employed, are non-toxic, chemically inert, and substantially non-immunogenic when used internally in the patient and which are substantially insoluble in physiologic liquids. Suitable biocompatible polymers include, by way of example, cellulose acetates (including cellulose diacetate), ethylene vinyl alcohol copolymers, hydrogels (e.g., acrylics), poly(C1-C6) acrylates, acrylate copolymers, polyalkyl alkacrylates wherein the alkyl and alk groups independently contain one to six carbon atoms, polyacrylonitrile, polyvinylacetate, cellulose acetate butyrate, nitrocellulose, copolymers of urethane/carbonate, copolymers of styrene/maleic acid, and mixtures thereof. Copolymers of urethane/carbonate include polycarbonates that are diol terminated which are then reacted with a diisocyanate such as methylene bisphenyl diisocyanate to provide for the urethane/carbonate copolymers.

Likewise, copolymers of styrene/maleic acid refer to copolymers having a ratio of styrene to maleic acid of from about 7:3 to about 3:7. Preferably, the biocompatible polymer is also non-inflammatory when employed in situ. The particular biocompatible polymer employed is not critical and is selected relative to the viscosity of the resulting polymer solution, the solubility of the biocompatible polymer in the biocompatible solvent, and the like. Such factors are well within the skill of the art.

The polymers of polyacrylonitrile, polyvinylacetate, poly(C1-C6) acrylates, acrylate copolymers, polyalkyl alkacrylates wherein the alkyl and alk groups independently contain one to six carbon atoms, cellulose acetate butyrate, nitrocellulose, copolymers of urethane/carbonate, copolymers of styrene/maleic acid and mixtures thereof typically will have a molecular weight of at least about 50,000 and more preferably from about 75,000 to about 300,000.

Preferred biocompatible polymers include cellulose diacetate and ethylene vinyl alcohol copolymer. In one embodiment, the cellulose diacetate has an acetyl content of from about 31 to about 40 weight percent. Cellulose diacetate polymers are either commercially available or can be prepared by art recognized procedures. In a preferred embodiment, the number average molecular weight, as determined by gel permeation chromatography, of the cellulose diacetate composition is from about 25,000 to about 100,000 more preferably from about 50,000 to about 75,000 and still more preferably from about 58,000 to 64,000. The weight average molecular weight of the cellulose diacetate composition, as determined by gel permeation chromatography, is preferably from about 50,000 to 200,000 and more preferably from about 100,000 to about 180,000. As is apparent to one skilled in the art, with all other factors being equal, cellulose diacetate polymers having a lower molecular weight will impart a lower viscosity to the composition as compared to higher molecular weight polymers. Accordingly, adjustment of the viscosity of the composition can be readily achieved by mere adjustment of the molecular weight of the polymer composition.

Ethylene vinyl alcohol copolymers comprise residues of both ethylene and vinyl alcohol monomers. Small amounts (e.g., less than 5 mole percent) of additional monomers can be included in the polymer structure or grafted thereon provided such additional monomers do not alter the implanting properties of the composition. Such additional monomers include, by way of example only, maleic anhydride, styrene, propylene, acrylic acid, vinyl acetate and the like.

Ethylene vinyl alcohol copolymers are either commercially available or can be prepared by art recognized procedures. Preferably, the ethylene vinyl alcohol copolymer composition is selected such that a solution of 8 weight-volume percent of the ethylene vinyl alcohol copolymer in DMSO has a viscosity equal to or less than 60 centipoise at 20° C. and more preferably 40 centipoise or less at 20° C. As is apparent to one skilled in the art, with all other factors being equal, copolymers having a lower molecular weight will impart a lower viscosity to the composition as compared to higher molecular weight copolymers. Accordingly, adjustment of the viscosity of the composition as necessary for catheter delivery can be readily achieved by mere adjustment of the molecular weight of the copolymer composition.

As is also apparent, the ratio of ethylene to vinyl alcohol in the copolymer affects the overall hydrophobicity/hydrophilicity of the composition which, in turn, affects the relative water solubility/insolubility of the composition as well as the rate of precipitation of the copolymer in an aqueous solution. In a particularly preferred embodiment, the copolymers employed herein comprise a mole percent of ethylene of from about 25 to about 60 and a mole percent of vinyl alcohol of from about 40 to about 75, more preferably a mole percent of ethylene of from about 40 to about 60 and a mole percent of vinyl alcohol of from about 40 to about 60.

The term “contrast agent” refers to a biocompatible (non-toxic) radiopaque material capable of being monitored during injection into a mammalian subject by, for example, radiography. The contrast agent can be either water soluble or water insoluble. Examples of water soluble contrast agents include metrizamide, iopamidol, iothalamate sodium, iodomide sodium, and meglumine. The term “water insoluble contrast agent” refers to contrast agents which are insoluble in water (i.e., has a water solubility of less than 0.01 milligrams per milliliter at 20° C.) and include tantalum, tantalum oxide and barium sulfate, each of which is commercially available in the proper form for in vivo use and preferably having a particle size of 10 μm or less. Other water insoluble contrast agents include gold, tungsten and platinum powders. Methods for preparing such water insoluble biocompatible contrast agents having an average particle size of about 10 μm or less are described below. Preferably, the contrast agent is water insoluble (i.e., has a water solubility of less than 0.01 mg/ml at 20° C.)

The term “encapsulation” as used relative to the contrast agent being encapsulated in the precipitate is not meant to infer any physical entrapment of the contrast agent within the precipitate much as a capsule encapsulates a medicament. Rather, this term is used to mean that an integral coherent precipitate forms which does not separate into individual components, for example into a copolymer component and a contrast agent component.

The term “biocompatible solvent” refers to an organic material liquid at least at body temperature of the mammal in which the biocompatible polymer is soluble and, in the amounts used, is substantially non-toxic. Suitable biocompatible solvents include, by way of example, dimethylsulfoxide, analogues/homologues of dimethylsulfoxide, ethanol, ethyl lactate, acetone, and the like. Aqueous mixtures with the biocompatible solvent can also be employed provided that the amount of water employed is sufficiently small that the dissolved polymer precipitates upon injection into a human body. Preferably, the biocompatible solvent is ethyl lactate or dimethylsulfoxide.

The compositions employed in the methods of this invention are prepared by conventional methods whereby each of the components is added and the resulting composition mixed together until the overall composition is substantially homogeneous. For example, sufficient amounts of the selected polymer are added to the biocompatible solvent to achieve the effective concentration for the complete composition. Preferably, the composition will comprise from about 2.5 to about 8.0 weight percent of the polymer based on the total weight of the composition and more preferably from about 4 to about 5.2 weight percent. If necessary, gentle heating and stirring ran be used to effect dissolution of the polymer into the biocompatible solvent, e.g., 12 hours at 50° C.

Sufficient amounts of the contrast agent are then optionally added to the biocompatible solvent to achieve the effective concentration for the complete composition. Preferably, the composition will comprise from about 10 to about 40 weight percent of the contrast agent and more preferably from about 20 to about 40 weight percent and even more preferably about 30 to about 35 weight percent. When the contrast agent is not soluble in the biocompatible solvent, stirring is employed to effect homogeneity of the resulting suspension. In order to enhance formation of the suspension, the particle size of the contrast agent is preferably maintained at about 10 μm or less and more preferably at from about 1 to about 5 μm (e.g., an average size of about 2 μm). In one preferred embodiment, the appropriate particle size of the contrast agent is prepared, for example, by fractionation. In such an embodiment, a water insoluble contrast agent such as tantalum having an average particle size of less than about 20 microns is added to an organic liquid such as ethanol (absolute) preferably in a clean environment. Agitation of the resulting suspension followed by settling for approximately 40 seconds permits the larger particles to settle faster. Removal of the upper portion of the organic liquid followed by separation of the liquid from the particles results in a reduction of the particle size which is confirmed under an optical microscope. The process is optionally repeated until a desired average particle size is reached.

The particular order of addition of components to the biocompatible solvent is not critical and stirring of the resulting suspension is conducted as necessary to achieve homogeneity of the composition. Preferably, mixing/stirring of the composition is conducted under an anhydrous atmosphere at ambient pressure. The resulting composition is heat sterilized and then stored preferably in sealed amber bottles or vials until needed.

Each of the polymers recited herein is commercially available but can also be prepared by methods well known in the art. For example, polymers are typically prepared by conventional techniques such as radical, thermal, UV, gamma irradiation, or electron beam induced polymerization employing, as necessary, a polymerization catalyst or polymerization initiator to provide for the polymer composition. The specific manner of polymerization is not critical and the polymerization techniques employed do not form a part of this invention. In order to maintain solubility in the biocompatible solvent, the polymers described herein are preferably not cross-linked.

In another particularly preferred embodiment of the nonaqueous solution, the biocompatible polymer composition can be replaced with a biocompatible prepolymer composition containing a biocompatible prepolymer. In this embodiment, the composition comprises a biocompatible prepolymer, an optional biocompatible water insoluble contrast agent preferably having an average particle size of about 10 μm or less and, optionally, a biocompatible solvent.

The term “biocompatible prepolymer” refers to materials which polymerize in situ to form a polymer and which, in the amounts employed, are non-toxic, chemically inert, and substantially non-immunogenic when used internally in the patient and which are substantially insoluble in physiologic liquids. Such a composition is introduced into the body as a mixture of reactive chemicals and thereafter forms a biocompatible polymer within the body. Suitable biocompatible prepolymers include, by way of example, cyanoacrylates, hydroxyethyl methacrylate, silicon prepolymers, and the like. The prepolymer can either be a monomer or a reactive oligomer. Preferably, the biocompatible prepolymer is also non-inflammatory when employed in situ.

Prepolymer compositions can be prepared by adding sufficient amounts of the optional contrast agent to the solution (e.g., liquid prepolymer) to achieve the effective concentration for the complete polymer composition. Preferably, the prepolymer composition will comprise from about 10 to about 40 weight percent of the contrast agent and more preferably from about 20 to about 40 weight percent and even more preferably about 30 weight percent. When the contrast agent is not soluble in the biocompatible prepolymer composition, stirring is employed to effect homogeneity of the resulting suspension. In order to enhance formation of the suspension, the particle size of the contrast agent is preferably maintained at about 10 μm or less and more preferably at from about 1 to about 5 μm (e.g., an average size of about 2 μm).

When the prepolymer is liquid (as in the case of polyurethanes), the use of a biocompatible solvent is not absolutely necessary but may be preferred to provide for an appropriate viscosity in the nonaqueous solution. Preferably, when employed, the biocompatible solvent will comprise from about 10 to about 50 weight percent of the biocompatible prepolymer composition based on the total weight of the prepolymer composition. When a biocompatible solvent is employed, the prepolymeric composition typically comprises from about 90 to about 50 weight percent of the prepolymer based on the total weight of the composition.

In a particularly preferred embodiment, the prepolymer is cyanoacrylate which is preferably employed in the absence of a biocompatible solvent. When so employed, the cyanoacrylate adhesive is selected to have a viscosity of from about 5 to about 20 centipoise at 20° C.

The particular order of addition of components is not critical and stirring of the resulting suspension is conducted as necessary to achieve homogeneity of the composition. Preferably, mixing/stirring of the composition is conducted under an anhydrous atmosphere at ambient pressure. The resulting composition is sterilized and then stored preferably in sealed amber bottles or vials until needed.

Specific embodiments of nonaqueous solutions suitable for use in the apparatus and methods of the invention are described in U.S. Pat. No. 5,667,767 dated Sep. 16, 1997, U.S. Pat. No. 5,580,568 dated Dec. 3, 1996 and U.S. Pat. No. 5,695,480 dated Dec. 9, 1997 and International Publication No. WO 97/45131 having an International Publication Date of Dec. 4, 1997, the entire contents of which are incorporated herein by this reference.

Other suitable implantable materials include any material capable of being delivered through a needle, solutions, suspensions, slurries, biodegradable or nonbiodegradable materials and two part or other mixtures. Exemplary implantable materials include injectable bioglass as described in Walker et al., “Injectable Bioglass as a Potential Substitute for Injectable Polytetrafluorethylene Particles”, J. Urol., 148:645-7, 1992, small particle species such as polytetrafluoroethylene (PTFE) particles in glycerine such as Polytef®, biocompatible compositions comprising discrete, polymeric and silicone rubber bodies such as described in U.S. Pat. Nos. 5,007,940, 5,158,573 and 5,116,387 to Berg, biocompatible compositions comprising carbon coated beads such as disclosed in U.S. Pat. No. 5,451,406 to Lawin, collagen and other biodegradable material of the type disclosed in U.S. Pat. No. 4,803,075 to Wallace et al., biocompatible materials such as disclosed in U.S. Pat. No. 6,296,607 to Milbocker, U.S. Pat. No. 6,524,327 to Spacek, and U.S. Publication Nos. 2002/0049363 and 2003/0135238 to Milbocker, and other known injectable materials.

A kit 96 for a use in treating a wall forming the upper portion of a gastrointestinal tract in a human body in accordance with the method of the present invention is shown schematically in FIG. 3. Kit 96 includes a package 97 made from any suitable material such as cardboard or plastic for carrying the contents thereof. An exemplary package 97, shown in FIG. 3, is a box formed from a bottom wall 98, four side walls 99 and a top wall 101. A portion of top wall 101 is cut away in FIG. 3 to reveal an internal space 102 formed by walls 98, 99 and 102. The contents of receptacle or package 97 are disposed in internal space 102.

Injection device 26 is carried by package 97 within internal space 102. As discussed above, the injection device 26 includes stylet 59 having needle member 61 and optional sleeve 62. A cap 106 is preferably attached to distal end portion 62b of the sleeve 62 for protecting users against undesirable punctures by needle 65 during storage and setup.

A reservoir or syringe 108, which can be included in supply 27, and a container or vial 109 of the implantable material referred to above can optionally be included, separately or together, within kit 96. Where vial 109, shown with cap 111 in FIG. 3, contains a solution for example of an implant-forming material, luer fitting portion 112 of the syringe 108 is removably coupleable to cap 111 of the vial 109. The luer fitting portion 112 of the syringe 108 is also removably coupleable to fitting 88 of finger-grippable element 86 of injection device 26. Additional optional components of kit 96 include a second reservoir, such as syringe 116, and a container of a biocompatible solvent such as DMSO in the form of vial 117. Vial 117 includes a cap 118 and syringe 116 has a luer fitting portion 119 removably coupleable to cap 118 of the vial 117. A third reservoir or syringe (not shown) and/or a vial of aqueous solution such as saline solution (not shown) can also be optionally included in kit 96.

Kit 96 can further include indifferent or return electrode 74, shown as a grounding pad 74, as well as cables 121 and 122. Cable 121 serves to electrically couple radio frequency supply and controller 28 to the injection device 26, and cable 122 serves to electrically couple the controller 28 to grounding pad 74.

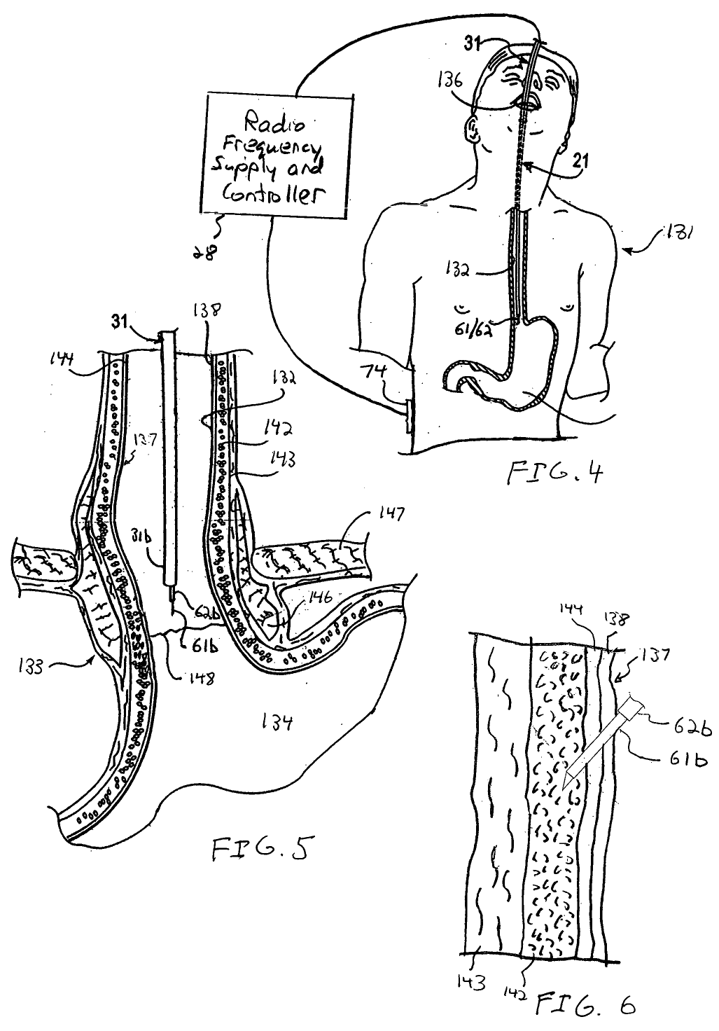

To assist in describing the utilization of the devices and practice of the method of the present invention, a portion of a mammalian body, in this case a human body 131, is shown 30 in FIGS. 4-6. Body 131 has an internal cavity in the form of the passage of the esophagus 132 extending through a lower esophageal sphincter 133 to a stomach 134. Such cavity is accessible by a natural body opening in the form of mouth 136 and is defined by a wall 137. Esophagus 132 is part of the gastrointestinal tract of body 131 that extends from mouth 136 to an anus (not shown). Wall 137 has a plurality of layers of tissue of respective thicknesses that includes at least first and second layers of tissue having respective first and second thicknesses. The esophageal mucosa 138 serves as the inner layer of the intraluminal wall 137 in the esophagus 132. Wall 137 has a muscle layer comprising layer of circular muscle 142 extending beneath mucosa layer 138 and layer of longitudinal muscle 143 beneath circular muscle 142. The muscle layers 142 and 143 each extend around the esophagus 132 and the stomach 134. Wall 137 further includes a submucosal layer or submucosal 144 extending between mucosa 138 and muscle layers 142 and 143. A submucosal space, that is a potential space, can be created between submucosal 144 and circular muscle layer 142 by the separation of layer 138 from muscle layer 142. In addition, as with any muscle, wall 137 includes an intramuscular potential space, that is a space which can be created intramuscularly by distension and separation of muscle fibers within a single muscle. Wall 137 has a depth or thickness which includes at least mucosal layer 138, submucosal layer 144, circular muscle layer 142 and longitudinal muscle layer 143. The phreno-esophageal ligament 146 and diaphragm 147 extend around the esophagus 132 above the lower esophageal sphincter 133. In the vicinity of the lower esophageal sphincter, as that term is used herein, includes at least the lower third of the esophagus 132, the squamous columnar junction 148, and the gastric cardia or upper portion of the stomach 188.

Although medical device 21 can be used in any number of procedures, in one preferred procedure the device is introduced into a natural body opening to access a vessel in the body, whether a passageway or an organ. In a further preferred procedure, device 21 can be utilized to deliver a fluid, composition or other material to a wall of a passageway within a mammalian body to treat the body and more particularly to the wall forming the gastrointestinal tract of a mammalian body. Particularly preferred procedures are described in U.S. Pat. Nos. 6,231,613, 6,234,955, 6,238,335, 6,248,058, 6,251,063, 6,251,064, 6,358,197, 6,540,789 and 6,595,910, the entire content of each of which is incorporated herein by this reference. The exemplary procedure utilized for describing the devices and methods of the present invention is the treatment of gastroesophageal reflux disease, for example as described in U.S. Pat. No. 6,251,063.

In operation and use of medical device 21 having injection device 26 in the method of the present invention, supply 27 is filled with an appropriate material in preparation of the procedure and coupled to the proximal extremity of needle member 61 by means of fluid connector 86. Controller 28 is also coupled to the proximal extremity of the needle member n a conventional manner and to grounding pad 74 placed in electrical contact with the exterior of human body 131. Probe 22 is prepared by connecting light cable 42 to light source 43 and attaching the proper eye piece 41 to handle 33. In addition, all other conventional attachments are applied to probe 22.

After the patient has been appropriately sedated or anesthetized, probe handle 33 is grasped by the physician to introduce distal extremity 31b of probe 22 into mouth 136 and advance insertion tube 31 down esophagus 132 to the vicinity of the lower esophageal sphincter 133. Insertion tube 31 has a length so that when distal extremity 31b is in the 1o vicinity of the tissue being treating, in this case in the vicinity lower esophageal sphincter 133, proximal extremity 31a is outside of body 131. The distal end portions or extremities 61b and 62b of injection device 26 are now inserted though side port 46 of insertion tube 31 and advanced until such end portions are in the vicinity of distal extremity 31b of the insertion tube 31. Distal extremity 31b of the insertion tube 31 is shown in the vicinity of lower esophageal sphincter 133 in FIGS. 4-6.

The physician causes sharpened tip 67 of needle 65 to penetrate or extend into wall 137 by moving needle member 61 and sleeve 62 closer to side port 46. The field of view of optical viewing device 23 permits the physician to observe the penetration of wall 137. The beveled tip of the needle 65 easily permits perforation of the tissue. Once the exposed portion 73 of the needle has been introduced into wall 137, desirably positioning of the needle 65 within the plurality of layers of the wall, for example in a desired layer of the wall 137, can be facilitated by supplying radio frequency energy from controller 28 to the needle 65 and measuring the impedance between the needle and the return electrode. The power supplied by controller 28 is sufficient to provide an impedance reading of the adjoining tissue, but not great enough to necrose the adjoining tissue. In this regard, any suitable power can be provided by the controller, for example two watts. Since impedance can distinguish different types of tissue, the exposed portion 73 of needle 65 can serve to locate the proper layer or location in the wall into which the implant-forming material is to be injected. The tissue resistance between needle 65 and grounding pad 74, that is impedance, is measured by and indicated at the controller 28 of such monopolar system of controller 28, needle 65 and grounding pad 74.

In one embodiment, needle 65 is configured so that the impedance measured by controller 28 ranges from one to 400 ohms and more specifically from 132 to 365 ohms, depending upon the layer of wall 137 in which the exposed portion 73 of the needle 65 is disposed. In general, more vascularized or wet layers or tissue, such as muscle layers 142 and 143, have a lower impedance than more dry layers of tissue, such as submucosal layer 144. The impedance measurement apparatus and procedure herein can thus serve to identify the desired layer or portion of tissue into which material from the needle 65 is to injected, and can also serve to indicate that the needle 65, including opening 71 therein, has been undesirably pushed through wall 137 and, for example, thus warn the operator of device 21 that no material should be injected from needle 65 into body 131 for fear of migration into undesired portions of the body.

Accurate placement of the needle 65 within wall 137, and thus accurate placement of the implant-forming material, is facilitated by the relatively small length of the distal portion of needle 65 which is exposed to form the exposed portion 73 of the needle 65. For example, by exposing one to two millimeters of the distal end of needle 65, the impedance measurement will also be across one to two millimeters, thus accurately targeting the proper layer of wall 137 for placement of the bollus of implant-forming material.

If the exposed portion or part 73 of needle 65 is not initially placed in the desired location within wall 137, the operator can use fluid connector 86 to advance or retract the needle 65 within the wall to a second location and measure again the impedance between the exposed part 73 and the return electrode to determine if the exposed part 73 of the needle 65 is desirably placed in the plurality of layers of the wall 137.

Once the needle 65 has been properly positioned, the physician causes an appropriate amount of injectable material to be introduced through needle 65 and into wall 137 to form at least one implant (not shown) in the desired layer of the wall. The injectable material can be deposited into any or all of the layers of wall 137, including between any of such layers. The implant can be of any suitable shape, for example an arcuate implant which extends around a portion or all of the wall as disclosed in U.S. Pat. No. 6,251,063. One or a plurality of implants can be formed in wall 137. Where a plurality of implants are formed, the implants can be disposed in any suitable configuration, for example circumferentially spaced apart, longitudinally spaced apart or circumferentially and longitudinally spaced apart. The implants can serve to augment the wall, bulk the wall, reduce the dispensability of muscle layers 142 and/or 143 of the wall, or serve any other purpose for treating the wall. When the ailment being treated is gastroesophageal reflux disease, the implant can serve to increase the competency of the lower esophageal sphincter 133.

It is appreciated that a bipolar system can be utilized for measuring tissue impedance and be within the scope of the present invention. In this regard, a return or bipolar or return electrode can be coupled to the interior of the mammalian body, for example by being located on distal extremity 61b of the needle member 61 proximal or distal of exposed portion 73 of the needle 65. Thus, the needle member 61 would include both the active electrode and return electrode, in positions spaced apart or separated by an insulating material or insulator. In another embodiment, a second needle can be introduced through probe 22 into wall 137, preferably in the vicinity of needle 65, for serving as a return electrode.

The invention, which includes needle 65 with layer 72 on at least a portion thereof can be used in any procedure for injecting a material into tissue of a body, whether the material be utilized for the formation of implants or any other purpose. For example, the invention can be used for treating fecal incontinence, such as disclosed in U.S. Pat. Nos. 6,251,063 and 6,595,910; for vascular occlusive therapy, such as treating hemorrhoids, varices and ulcers as disclosed in U.S. Pat. No. 6,234,955; for treating tracheo-esophageal fistulas, such as disclosed in U.S. Pat. No. 6,248,058 and for treating morbid obesity, such has disclosed in U.S. Pat. No. 6,540,789. The invention facilitates the accurate placement of the material in the desired layer or between the desired layers of tissue.

As can be seen from the foregoing, a medical device for the delivery of an injectable material into the tissue of a mammalian body has been provided which facilitates accurate placement of the material in the targeted tissue. The device permits the operator to more clearly distinguish between tissue layers. The injected material can be used for the formation of implants. An injection device and method can be provided.

A kit for providing an implant in tissue having a plurality of layers with respective thicknesses in a mammalian body can be provided and comprises a package, a needle carried by the package and having a distal portion, a layer of insulating material extending around the needle but exposing a length of the distal portion of the needle, the length of the exposed distal portion of the needle being sized as a function of the thicknesses of the plurality of layers of tissue to facilitate placement of the distal portion of the needle in the plurality of layers, and a container of implantable material carried by the package for providing an implant in the tissue. The implantable material can be a nonaqueous solution for forming a nonbiodegradable solid in the tissue. The layer of insulating material can be nonmovably secured to the needle. The kit of can further comprise a container of a biocompatible solvent carried by the package.

1. An apparatus for use with a radio frequency generator and an indifferent electrode to treat tissue having a plurality of layers with respective thicknesses in a mammalian body comprising a needle having a distal portion and being adapted for coupling to the radio frequency generator and a layer of insulating material extending around the needle but exposing a length of the distal portion of the needle, the length of the exposed distal portion of the needle being sized as a function of the thicknesses of the plurality of layers of tissue to facilitate placement of the distal portion of the needle in the plurality of layers by monitoring impedance between the needle and the indifferent electrode.

2. The apparatus of claim 1 wherein the plurality of layers includes first and second layers having respective first and second thicknesses, the first thickness being less than the second thickness and the length of the exposed distal portion of the needle being less than the first thickness.

3. The apparatus of claim 1 wherein the needle has a diameter ranging from 0.5 to 3.0 millimeters and the length of the exposed distal portion of the needle ranges from 0.2 to 2.0 millimeters.

4. The apparatus of claim 3 wherein the needle has a diameter of approximately 0.5 millimeters and the length of the exposed distal portion of the needle is approximately 0.5 millimeters.

5. The apparatus of claim 1 wherein the layer of insulating material has a thickness ranging from 0.0005 to 0.0020 inches.

6. The apparatus of claim 1 further comprising a flexible elongate tubular member for introducing the needle into the mammalian body, the needle being slidably disposed in the flexible elongate tubular member.

7. The apparatus of claim 6 wherein the flexible elongate tubular member has proximal and distal extremities and a sufficient length so that when the distal extremity is in the vicinity of the gastroesophageal sphincter the proximal extremity is outside of the body.

8. An apparatus for treating tissue of a mammalian body having a plurality of layers with respective thicknesses comprising a needle having proximal and distal portions, a radio frequency generator coupled to the proximal portion of the needle for supplying radio frequency energy to the needle, an indifferent electrode coupled to the radio frequency generator and adapted to couple to the mammalian body, a layer of insulating material extending around the distal portion of the needle but exposing part of the distal portion of the needle, the exposed part being sized as a function of the thicknesses of the plurality of layers of tissue to facilitate placement of the distal portion of the needle in the plurality of layers by monitoring impedance between the needle and the indifferent electrode.

9. The apparatus of claim 8 wherein the indifferent electrode is a grounding pad.

10. The apparatus of claim 8 wherein the indifferent electrode is a bipolar electrode.

11. The apparatus of claim 8 further comprising a flexible elongate tubular member for introducing the needle into the mammalian body, the needle being slidably disposed in the flexible elongate tubular member.

12. A method for treating tissue having a plurality of layers in a mammalian body comprising providing a needle having a distal portion and a layer of insulating material extending around the distal portion but exposing part of the distal portion, coupling a return electrode to the mammalian body, extending the needle into the tissue, supplying radio frequency energy to the needle, measuring the impedance between the needle and the return electrode to determine if the exposed part of the distal portion of the needle is desirably placed in the plurality of layers and introducing a material through the needle into the tissue.

13. The method of claim 12 further comprising moving the needle further into the tissue and measuring again the impedance between the needle and the return electrode to determine if the exposed part of the distal portion of the needle is desirably placed in the plurality of layers.

14. The method of claim 12 wherein the coupling step includes coupling a return electrode to the exterior of the mammalian body.

15. The method of claim 12 wherein the coupling step includes coupling a return electrode to the interior of the mammalian body.

16. The method of claim 12 further comprising forming an implant in the tissue from the material.

17. The method of claim 12 wherein the extending step includes extending the needle into the tissue of a wall of an esophagus in the vicinity of the gastroesophageal sphincter.

18. The method of claim 12 wherein the extending step includes extending the needle into the tissue of a wall of an anal canal.

19. The method of claim 12 wherein the extending step includes extending the needle into the tissue of a wall forming the stomach.

20. The method of claim 12 wherein the extending step includes extending the needle through the gastrointestinal tract into tissue of a wall forming the gastrointestinal tract.
